(19) United States
(12) Patent Application Publication
(10) Pub. No.: US 2026/0073593 A1
(43) Pub. Date: Mar. 12, 2026

(54) MEDICAL IMAGE PROCESSING APPARATUS, MEDICAL IMAGE PROCESSING SYSTEM, MEDICAL IMAGE PROCESSING METHOD, AND STORAGE MEDIUM

(71) Applicant: Konica Minolta, Inc., Tokyo (JP)

(72) Inventors: Naoki Hayashi, Tokyo (JP); Yozo Okubo, Tokyo (JP); Hirosuke Shirakawa, Tokyo (JP)

(73) Assignee: Konica Minolta, Inc., Tokyo (JP)

(21) Appl. No.: 19/317,887

(22) Filed: Sep. 3, 2025

(30) Foreign Application Priority Data
Sep. 10, 2024 (JP) 2024-156506

(51) Int. Cl. G06T 11/60; G06T 7/00; G16H 30/40

(52) U.S. Cl. CPC G06T 11/60; G06T 7/0016; G16H 30/40; G06T 2207/20224; G06T 2207/30008

(57) ABSTRACT

A medical image processing apparatus includes a hardware processor that performs temporal subtraction (TS) processing, based on medical images on which bone suppression (BS) processing has been performed to obtain a BSTS image. In the BSTS image, information indicating that the BS processing has been performed is visibly embedded.

### CROSS-REFERENCE TO RELATED APPLICATIONS

The entire disclosure of Japanese Patent Application No. 2024-156506 filed on Sep. 10, 2024, is incorporated herein by reference in its entirety.

### Technical Field

The present disclosure relates to a medical image processing apparatus, a medical image processing system, a medical image processing method, and a storage medium.

### Background of the Invention

Conventionally, temporal subtraction (TS) processing has been performed to check temporal changes, such as progress of a lesion. In TS processing, two medical images captured at different times are used: the positions of the structure captured in the medical images are aligned, and after alignment, subtraction processing is performed to extract information on temporal changes only.

However, in TS processing, artifacts often occur even if the position alignment, warping processing for alignment, and so forth have been performed. This is because the two medical images have been obtained under different conditions, such as the state of the patient and the imaging environment.

To deal with this, image processing for reducing artifacts is performed before and after TS processing.

For example, according to Japanese Unexamined Patent Publication No. 2004-194357, artifacts are reduced by blurring edge portions of bones in medical images using a blurring mask to generate blurred images, and TS processing is performed between the blurred images.

Further, according to Japanese Unexamined Patent Application Publication (Translation of PCT Application) No. 2017-510427, bone suppression (BS) processing is performed for attenuating bones so that a doctor can easily view a lung-field portion overlapping with ribs and so forth when diagnosing a lung-field region using chest medical images. By performing BS processing as preprocessing of TS processing, artifacts can be reduced.

After BS processing, an annotation is added to the medical image (BS image) to indicate that BS processing has been performed. Herein, “adding an annotation” means embedding the annotation in a BS image such that the image data is replaced with the annotation. There are generally two methods of adding an annotation: one is to embed the annotation in the image; and the other is to save the annotation as supplementary information of the image and to superpose and display the annotation on the image. However, when the storage of the supplementary information and the display are in different systems, the supplementary information cannot be read. Therefore, the supplementary information is often embedded in the image.

When TS processing is performed after BS processing, in TS processing, the embedded parts may overlap and disappear or may blur due to the deviation in positions of embedded parts in the medical image (BSTS image) on which BS processing and TS processing have been performed. In such a case, the annotation becomes less visible, or the doctor may not be able to distinguish between a BSTS image and a TS image on which only TS processing has been performed because the TS image and BSTS image are similar.

Even if a doctor does not know that the position alignment and warping processing have been performed on medical images, the doctor can make diagnosis without any trouble. However, if the doctor does not know that BS processing has been performed, the doctor may be led to a misdiagnosis because the doctor does not know that a structure, such as bones, has been attenuated.

An object of the present disclosure is to correctly display an annotation in a BSTS image.

### SUMMARY OF THE INVENTION

In order to solve the above problem, according to an aspect of the present disclosure, there is provided a medical image processing apparatus including a hardware processor that performs temporal subtraction (TS) processing, based on medical images on which bone suppression (BS) processing has been performed to obtain a BSTS image, wherein, in the BSTS image, information indicating that the BS processing has been performed is visibly embedded.

According to another aspect of the present disclosure, there is provided a medical image processing system including a hardware processor and a display, wherein: the hardware processor performs TS processing, based on medical images on which BS processing has been performed to obtain a BSTS image, in the BSTS image, information indicating that the BS processing has been performed is visibly embedded, and the display displays the information indicating that the BS processing has been performed.

According to another aspect of the present disclosure, there is provided a medical image processing method for a medical image processing apparatus including a hardware processor that performs TS processing, based on medical images on which BS processing has been performed to obtain a BSTS image, the method including visibly embedding, in the BSTS image, information indicating that the BS processing has been performed.

According to another aspect of the present disclosure, there is provided a non-transitory computer-readable storage medium storing a program that causes a computer of a medical image processing apparatus to: perform TS processing, based on medical images on which BS processing has been performed to obtain a BSTS image and visibly embed, in the BSTS image, information indicating that the BS processing has been performed.

### BRIEF DESCRIPTION OF DRAWINGS

The advantages and features provided by one or more embodiments of the disclosure will become more fully understood from the detailed description given hereinafter and the appended drawings which are given by way of illustration only, and thus are not intended as a definition of the limits of the present disclosure, and wherein:

FIG. 1 is a block diagram illustrating a configuration of a medical image processing system;

FIG. 2 is a flowchart of BSTS processing;

FIG. 3 is an image diagram of TS processing in BSTS processing;

FIG. 4 is an image diagram of TS processing in BSTS processing;

FIG. 5 is a flowchart of BSTS processing; and

FIG. 6 is an image diagram of TS processing in BSTS processing.

### DETAILED DESCRIPTION

Hereinafter, an embodiment of the present disclosure is described below with reference to the drawings. However, the embodiment described below has various limitations which are technically preferable for carrying out the present disclosure. Therefore, the technical scope of the present disclosure is not limited to the following embodiment and illustrated examples.

### Medical Image Processing System 100

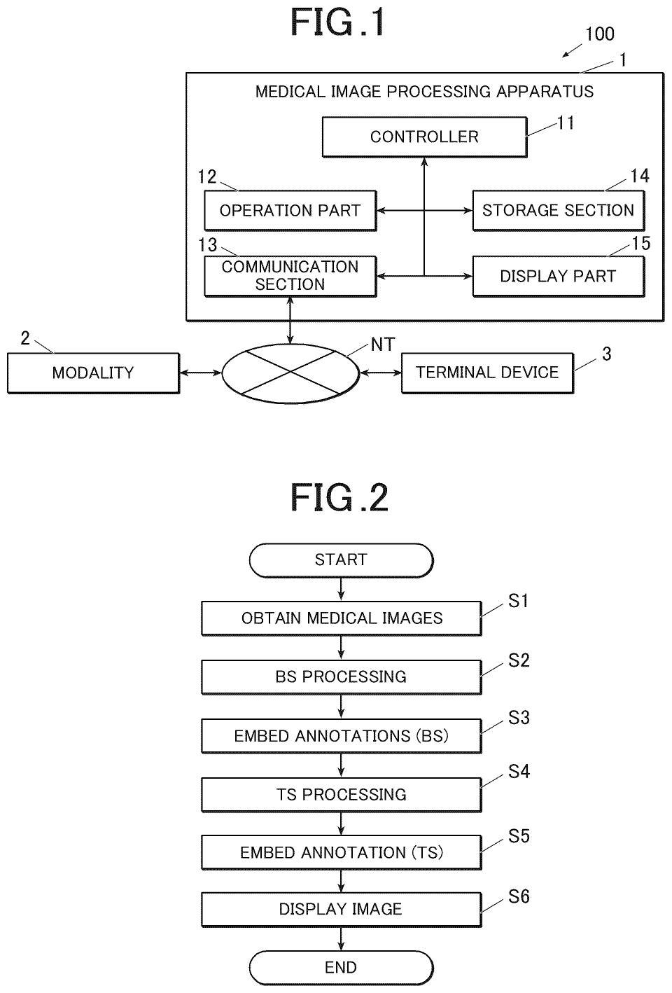

First, a configuration of a medical image processing system 100 will be described with reference to FIG. 1. As illustrated in FIG. 1, the medical image processing system 100 includes a medical image processing apparatus 1, a modality 2, and a terminal device 3 that are connected via a communication network NT, such as a local area network (LAN).

The medical image processing apparatus 1 is an information processing apparatus that performs BS processing (bone suppression processing) on medical images, performs TS processing (temporal subtraction processing) on medical images, and performs TS processing after BS processing on medical images. Specifically, the medical image processing apparatus 1 is an information processing apparatus, such as a cloud server or an on-premise server.

The modality 2 is an apparatus that obtains or manages medical images. Specifically, the modality 2 is any type of medical imaging apparatuses that takes medical images, such as an X-ray imaging apparatus (DR and CR), an ultrasonography (US) apparatus, a computed tomography (CT) apparatus, and a magnetic resonance imaging (MRI) apparatus or any type of medical image management apparatus that manages medical images, such as a radiology information system (RIS) and a picture archiving and communication system (PACS).

The terminal device 3 is for a user, such as a doctor, to check medical images transmitted from the medical image processing apparatus 1 or the modality 2. The terminal device 3 includes a display part (not illustrated).

### Medical Image Processing Apparatus 1

A configuration of the medical image processing apparatus 1 according to the present embodiment will be described with reference to FIG. 1. FIG. 1 is a block diagram illustrating the configuration of the medical image processing apparatus 1.

As shown in FIG. 1, the medical image processing apparatus 1 includes a controller 11 (hardware processor), an operation part 12, a communication section 13, a storage section 14, and a display part 15.

The controller 11 includes a central processing unit (CPU) and a random-access memory (RAM). The CPU of the controller 11 reads various programs stored in the storage section 14, loads the programs in the RAM, executes various processes in accordance with the loaded programs, and controls operation of each section of the medical image processing apparatus 1.

The controller 11 functions as a processing section (first processing section) that performs TS processing on medical images on which BS processing has been performed.

The controller 11 functions as a second processing section that performs TS processing on medical images on which BS processing has not been performed.

The medical images are obtained by imaging the same site of the same patient.

The operation part 12 includes a keyboard having cursor keys, number input keys, and various function keys, a pointing device such as a mouse, and a touch screen layered on the surface of the display part 15. The operation part 12 is operable by an operator. The operation part 12 outputs various signals based on an operation performed by the operator to the controller 11.

The communication section 13 can transmit and receive various signals and various data to and from other devices coupled via the communication network N.

The storage section 14 includes a nonvolatile semiconductor memory and/or a hard disk and stores various programs to be executed by the controller 11, parameters required for execution of the programs, various data, and so forth.

The display part 15 includes a monitor such as a liquid crystal display (LCD) and displays various screens in accordance with instructions of display signals input from the controller 11.

### BSTS Processing: Case 1

Next, BSTS processing in the present embodiment will be described with reference to FIG. 2.

In BSTS processing, BS processing is performed on medical images, and thereafter TS processing is performed on the medical images. In the following description of BSTS processing, two medical images are used in BSTS processing. Two medical images are obtained by imaging the chest of an identical patient.

In the following. “embedding an annotation” means replacing image data with the annotation.

First, the controller 11 obtains two medical images from the modality 2 via the communication section 13 (step S1).

Next, the controller 11 performs BS processing on the two medical images to generate BS images (step S2).

Next, the controller 11 embeds, in each BS image, an annotation indicating that BS processing has been performed (step S3).

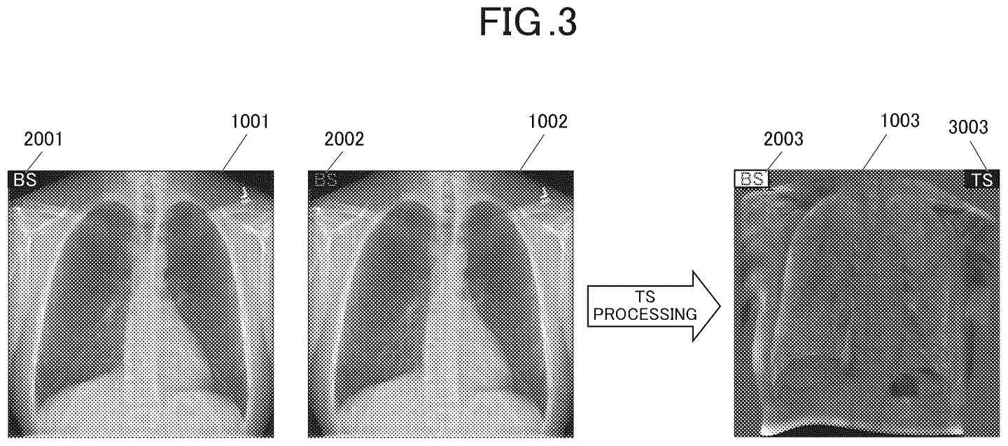

For example, as shown in FIG. 3, the controller 11 may embed annotations having different pixel values in the two BS images.

In the BS image 1001, an annotation 2001 is embedded at the upper left of the image.

In the BS image 1002, an annotation 2002 is embedded at the upper left of the image.

The annotation 2001 and the annotation 2002 have different pixel values. Specifically, the annotation 2001 is the white “BS” on a black background, and the annotation 2002 is the gray “BS” on a black background.

In the case of FIG. 3, the annotations have the same name (“BS”) and are embedded at the same position.

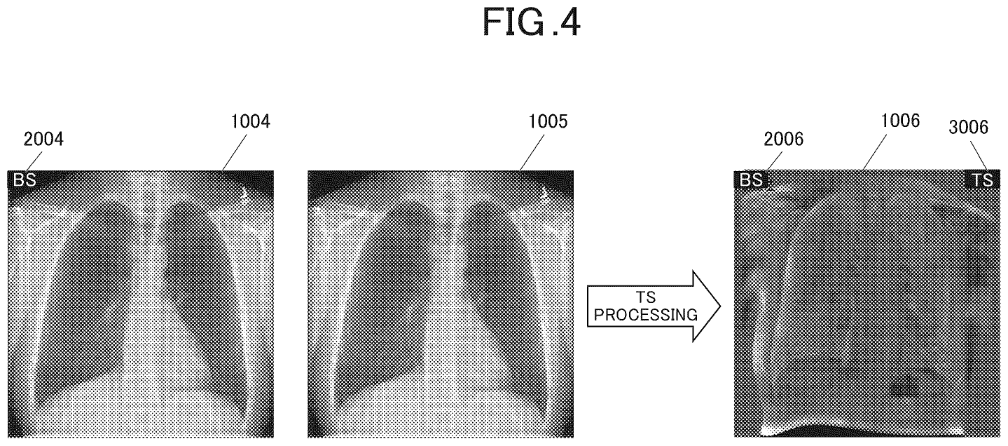

For another example, as illustrated in FIG. 4, the controller 11 may embed an annotation only in one image and may not embed an annotation in the other image.

In the BS image 1004, an annotation 2004 is embedded at the upper left of the image.

In the BS image 1005, no annotation is embedded.

When three or more medical images are used, the controller 11 may embed an annotation in only one BS image.

Next, the controller 11 performs TS processing on the two BS images to generate a BSTS image (step S4). In TS processing, parts of the images where the annotations are embedded are also subjected to TS processing.

For example, as shown in FIG. 3, the controller 11 performs TS processing by obtaining the difference between the two BS images 1001 and 1002 to generate a BSTS image 1003.

At this time, the difference between the annotations 2001 and 2002 embedded in the two images 1001 and 1002 is also obtained. Since the two annotations 2001 and 2002 have different pixel values, the parts where the annotations are embedded also have a difference.

Accordingly, in the BSTS image 1003, the annotation 2003 is visibly embedded at the upper left of the image as information indicating that BS processing has been performed.

Thus, the annotation can be correctly displayed in the BSTS image.

The controller 11 may determine whether or not the annotation after TS processing is at a specified display position or conforms to a specified display format. When the annotation is at the specified display position or conforms to the specified display format, the controller 11 may maintain the annotation as it is; whereas when not, the controller 11 may rewrite the annotation. For example, information embedded in the BSTS images in different display formats decreases usability. In such a case, the area where an annotation is embedded (annotation area) may be rewritten into an annotation indicating BS processing. Specifically, the controller 11 determines, using machine learning or the like, whether the result of TS processing is visible. When the visibility is bad, the controller 11 rewrites the annotation into a predetermined display format, for example.

Further, in the above case, the controller 11 may remove the annotation parts and newly add annotations before TS processing.

The same applies to a case where the annotations are drawn at different positions in addition to the case where the annotations have different pixel values. If the annotations are drawn at different (shifted) positions, the annotations are processed in a shifted state in TS processing, so that the characters thereof cannot be read. To deal with this, processing of step S14 or step S15, which is described later, may be performed on the annotation areas.

Further, as shown in FIG. 4, the controller 11 performs TS processing by obtaining the difference between the two BS images 1004 and 1005 to generate a BSTS image 1006.

Herein, the difference at the annotation 2004 embedded in the image 1004 is also obtained. Since only the image 1004 has the annotation 2004, the difference occurs at the part where the annotation 2004 is embedded.

Accordingly, in the BSTS image 1006, the annotation 2006 is visibly embedded at the upper left of the image as information indicating that BS processing has been performed.

Thus, the annotation can be correctly displayed in the BSTS image.

Next, the controller 11 embeds, in the BSTS image, an annotation indicating that TS processing has been performed (step S5).

For example, as shown in FIG. 3, the controller 11 may embed an annotation 3003 in the BSTS image 1003. For another example, as shown in FIG. 4, the controller 11 may embed an annotation 3006 in the BSTS image 1006.

Next, the controller 11 displays the BSTS image on the display part 15 or the display part of the terminal device 3 and ends BSTS processing (step S6).

### BSTS Processing: Case 2

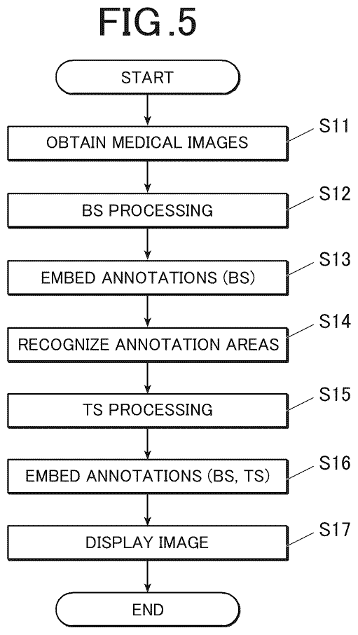

Next, BSTS processing in the present embodiment will be described with reference to FIG. 5.

In the following description of BS processing, two medical images are used in BSTS processing. Two medical images are obtained by imaging the chest of an identical patient.

First, the controller 11 obtains two medical images from the modality 2 via the communication section 13 (step S11).

Next, the controller 11 performs BS processing on the two medical images to generate BS images (step S12).

Next, the controller 11 embeds, in each BS image, an annotation indicating that BS processing has been performed (step S13).

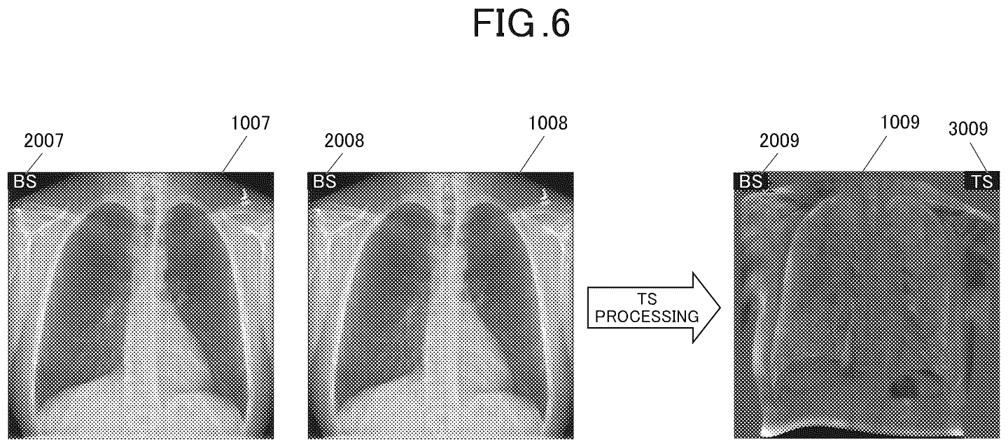

For example, as shown in FIG. 6, the controller 11 may embed annotations 2007 and 2008 in the two BS images 1007 and 1008, respectively.

In the BS image 1007, the annotation 2007 is embedded at the upper left of the image.

In the BS image 1008, the annotation 2008 is embedded at the upper left of the image.

In the case of FIG. 6, the annotations are embedded at the same position.

Next, the controller 11 recognizes the area (annotation area) in which the annotation is embedded in the BS image (step S14).

Specifically, the controller 11 may recognize the annotation area by using various analysis models, such as a machine learning model trained to recognize an annotation area in an image.

Further, when the position of embedding an annotation in an image is determined beforehand, the controller 11 may recognize the position as an annotation area.

Next, the controller 11 performs TS processing on the two BS images by a predetermined method to generate a BSTS image (step S15).

For example, the controller 11 may delete image parts corresponding to the annotation areas recognized in step S4 from the two BS images 1007 and 1008 and then perform TS processing.

For another example, the controller 11 may perform TS processing without obtaining the difference between the annotation areas recognized in step S14. As described later, the annotation of one of the two BS images 1007 and 1008 may be preferentially used for the annotation area.

Next, the controller 11 embeds, in the BSTS image, an annotation indicating that BS processing and TS processing have been performed (step S16).

For example, the controller 11 may embed the annotation 2009 in the annotation area recognized in step S4 as information indicating that BS processing has been performed.

Further, for example, the controller 11 may preferentially use the image of the annotation area of either of the BS images. Specifically, the controller 11 may prioritize the BS image 1007 and embed the annotation 2007 as the annotation 2009.

Further, for example, as shown in FIG. 6, the controller 11 may embed an annotation 3009 indicating TS processing in the medical image.

Thus, in the BSTS image 1009, the annotation 2009 is embedded in the upper left of the image as information indicating that BS processing has been performed. Thus, the BSTS image visibly indicates that BS processing has been performed.

Thus, the annotation can be correctly displayed in the BSTS image.

Next, the controller 11 displays the BSTS image on the display part 15 or the display part of the terminal device 3 and ends BSTS processing (step S17).

### Others

Although BSTS processing has been described above, based on the case where BSTS processing is performed using two medical images, the present disclosure is not limited to this example. Three or more medical images may be used.

Further, the controller 11 may display an annotation indicating that BS processing and/or TS processing have been performed in an area other than the area showing the structure of the subject. In this case, the controller 11 may automatically adjust a mark, wording, and/or size of the annotation, depending on the determined position and area where the annotation is displayed. For example, as illustrated in FIG. 3, the controller 11 may put the annotation 2001 in an area of the BS image 1001 other than portions of the subject.

Further, the annotation indicating that BS processing has been performed or the annotation indicating that TS processing has been performed may be drawn with white on a black background, as illustrated in FIGS. 3, 4, and 6. The background color and the character color are not limited to this example.

Further, in the BSTS processing flow illustrated in FIG. 2 and FIG. 5, TS processing is performed after BS processing. However, the controller 11 may obtain BS images on which BS processing has been performed beforehand from the storage section 14 and perform TS processing.

Further, information indicating that BS processing has been performed and information indicating that TS processing has been performed may be embedded such that they are distinguishable from each other. For example, in the examples of FIGS. 3, 4, and 6, the character information is differentiated as “BS” and “TS” so that they are distinguishable.

The information indicating that BS processing has been performed and the information indicating that TS processing has been performed may be embedded so as not to overlap each other. For example, in the examples of FIGS. 3, 4, and 6, the controller 11 embeds the annotation indicating “BS” in the upper left of the medical image and the annotation indicating “TS” in the upper right of the medical image.

The controller 11 can also perform TS processing on medical images on which BS processing is not performed. In this case, the controller 11 may embed an annotation indicating “TS” in the upper right of the medical image as information indicating that TS processing has been performed, similarly to the examples of FIGS. 3, 4, and 6. For example, an annotation indicating “TS” may be embedded with white information on a black background. Further, for example, the annotation indicating that TS processing has been performed may be displayed in an area other than the area showing the structure of the subject.

As described above, the controller 11 may display the annotation indicating “TS” and the annotation indicating “BS” in a distinguishable manner.

In all of the above examples, the annotation of BS is located at the upper left of the image. In a case where the annotations indicating “BS” are shown at different positions in first and second BS images (e.g., at the upper left and the lower right), the annotations indicating “BS” may not be shown on the two positions but may be rewritten into one position. In such a case, for example, the controller 11 specifies the positions of the annotations indicating “BS” by machine learning or the like, erases each annotation, and then re-embeds annotations at only one predetermined position.

Re-embedding of annotations as described above may be performed not only in a case where annotations are displayed at multiple positions or in a case where displayed annotations are invisible, but also in a case where annotations are not displayed at a position suitable for the final image. For example, there may be a case where “BS” is embedded in the upper center of a past image (first image) and the difference between the past image and the latest image (second image) is obtained. Since the upper center portion overlaps with the neck of the subject, the user may want to change the position of the annotation to the upper left in terms of visibility.

### Effects

As described above, the medical image processing apparatus 1 includes the processing section (controller 11) that performs temporal subtraction (TS) processing, based on medical images on which bone suppression (BS) processing has been performed. In the image obtained by TS processing, information indicating that BS processing has been performed is visibly embedded.

Thus, the annotation can be correctly displayed in the BSTS image.

The medical image processing system 100 further includes a first processing section (controller 11) that performs time-lapse subtraction processing on the plurality of medical images on which the bone attenuation processing has been performed and a display part (the display part of the terminal device 3), wherein the information indicating that the bone attenuation processing has been performed is embedded in the image after the time-lapse subtraction processing and is input in a manner recognizable from the image after the time-lapse subtraction processing, and the display part displays the information indicating that the bone attenuation processing has been performed.

Thus, the annotation can be correctly displayed in the BSTS image.

The medical image processing method by the medical image processing apparatus 1 including the processing section (controller 11) that performs the time-lapse subtraction processing on a plurality of medical images obtained by performing the bone-weakening processing on the plurality of medical images includes steps (step S5 and step S16) of embedding information indicating that the bone-weakening processing has been performed in the images after the time-lapse subtraction processing and inputting the information so as to be visually recognizable from the images after the time-lapse subtraction processing.

Thus, the annotation can be correctly displayed in the BSTS image.

Furthermore, the program causes the computer of the medical image processing apparatus 1 to function as a processing section (the controller 11) to perform time-lapse subtraction processing on a plurality of medical images obtained by performing bone attenuation processing on the plurality of medical images, so that information indicating that bone attenuation processing has been performed is input in a manner such that the information is embedded in the images after the time-lapse subtraction processing and is visibly recognizable from the images after the time-lapse subtraction processing.

Thus, the annotation can be correctly displayed in the BSTS image.

Although the present disclosure has been described in detail based on the embodiment, the present disclosure is not limited to the above-described embodiment. The embodiment can be modified without departing from the spirit and scope of the invention.

Although an example in which a semiconductor nonvolatile memory, a hard disk, or the like is used as a computer-readable medium of the program according to the present invention has been disclosed in the above description, the present invention is not limited to this example.

As other computer-readable media, a nonvolatile memory, such as a flash memory, and a portable recording medium, such as a CD-ROM, are also applicable.

As a medium for providing data of the program according to the present disclosure via a communication line, a carrier wave is also applied to the present disclosure.

Although embodiments of the present disclosure have been described and illustrated in detail, the disclosed embodiments are made for purposes of illustration and example only and not limitation. The scope of the present disclosure should be interpreted by terms of the appended claims.

1. A medical image processing apparatus comprising a hardware processor that performs temporal subtraction (TS) processing, based on medical images on which bone suppression (BS) processing has been performed to obtain a BSTS image,
wherein, in the BSTS image, information indicating that the BS processing has been performed is visibly embedded.

2. The medical image processing apparatus according to claim 1, wherein the information indicating that the BS processing has been performed is embedded in the BSTS image by replacing part of image data of the BSTS image with the information.

3. The medical image processing apparatus according to claim 1, wherein:
the medical images on which the BS processing has been performed include a first image and a second image,
in the first image, first information indicating that the BS processing has been performed is embedded,
in the second image, second information indicating that the BS processing has been performed is embedded, and
the first information and the second information have different pixel values.

4. The medical image processing apparatus according to claim 1, wherein:
the medical images on which the BS processing has been performed include a first image and a second image, and
the information indicating that the BS processing has been performed is embedded in only either the first image or the second image.

5. The medical image processing apparatus according to claim 1, wherein the information indicating that the BS processing has been performed is embedded in the BSTS image after the TS processing.

6. The medical image processing apparatus according to claim 1, wherein the information indicating that the BS processing has been performed is shown in an area other than an area showing a structure of a subject.

7. The medical image processing apparatus according to claim 1, wherein the information indicating that the BS processing has been performed is shown in white on a black background.

8. The medical image processing apparatus according to claim 1, wherein in the BSTS image, information indicating that the TS processing has been performed is visibly embedded.

9. The medical image processing apparatus according to claim 8, wherein the information indicating that the TS processing has been performed is shown in white on a black background.

10. The medical image processing apparatus according to claim 8, wherein the information indicating that the BS processing has been performed and the information indicating that the TS processing has been performed are embedded to be distinguishable from each other.

11. The medical image processing apparatus according to claim 8, wherein the information indicating that the BS processing has been performed and the information indicating that the TS processing has been performed are embedded not to overlap each other.

12. A medical image processing system comprising a hardware processor and a display, wherein:
the hardware processor performs TS processing, based on medical images on which BS processing has been performed to obtain a BSTS image,
in the BSTS image, information indicating that the BS processing has been performed is visibly embedded, and
the display displays the information indicating that the BS processing has been performed.

13. The medical image processing system according to claim 12, wherein:
the hardware processor performs TS processing, based on medical images on which the BS processing is not performed to obtain a TS image, and
the display displays (i) information indicating that the BS processing is not performed and that the TS processing has been performed and (ii) the information indicating that the BS processing has been performed so as to be distinguishable from each other.

14. The medical image processing system according to claim 13, wherein the information indicating that the BS processing has been performed is information indicating that both the BS processing and the TS processing have been performed.

15. The medical image processing system according to claim 13, wherein:
the display displays, on the TS image, the information indicating that the BS processing is not performed and that the TS processing has been performed, and
the display displays, on the BSTS image, information indicating that both the BS processing and the TS processing have been performed.

16. A medical image processing method for a medical image processing apparatus including a hardware processor that performs TS processing, based on medical images on which BS processing has been performed to obtain a BSTS image, the method comprising visibly embedding, in the BSTS image, information indicating that the BS processing has been performed.

17. A non-transitory computer-readable storage medium storing a program that causes a computer of a medical image processing apparatus to:
perform TS processing, based on medical images on which BS processing has been performed to obtain a BSTS image and
visibly embed, in the BSTS image, information indicating that the BS processing has been performed.
